(19) United States
(12) Patent Application Publication
(10) Pub. No.: US 2014/0114272 A1
(43) Pub. Date: Apr. 24, 2014

(54) Method And Apparatus For Forming An Adjustable Pant-Like Disposable Undergarment With Fully Severed Front Panel With Line Of Weakness

(71) Applicant: KIMBERLY-CLARK WORLDWIDE, INC., Neenah, WI (US)

(72) Inventors: Bradley William Schoon, Oshkosh, WI (US); Jason Andrew Verboomen, Appleton, WI (US); Robert Lee Popp, Greenville, WI (US); Patrick August Lorsung, Amherst, WI (US)

(73) Assignee: Kimberly-Clark Worldwide, Inc., Neenah, WI (US)

(21) Appl. No.: 13/658,879

(22) Filed: Oct. 24, 2012

(51) Int. Cl. A61F 13/56; B26F 3/00

(52) U.S. Cl. 604/386; 225/2

(57) ABSTRACT

A method and apparatus for producing a pre-fastened adjustable pant-like disposable absorbent undergarment including a fully severed front body panel is disclosed. The method includes the steps of providing a moving web and first creating a line of weakness on the moving web at a weakening station to define a leading portion of the moving web and a trailing portion of the moving web connected at the line of weakness. The moving web then passes through an attachment assembly station to bridge the connected leading portion and trailing portion together with a fastener assembly that extends over the line of weakness. Finally, the moving web is then passed through a web breaking station to completely separate the leading portion and trailing portion of the moving web at the line of weakness, such that the leading portion and trailing portion remains bridged together by the fastener assembly.

### BACKGROUND

Pant-like disposable undergarments for absorbing human discharges can appear similar in size and shape to regular cloth underwear which is designed to be laundered and reused. A disposable absorbent undergarment is intended to be worn by persons, including infants, toddlers, or adults, and is designed for a single or temporary use and is meant to be disposed of after being used once instead of being laundered or dry cleaned for re-use. Some examples of disposable undergarments include infant diapers, training pants, adult incontinence garments, feminine pants, etc.

Some pant-like disposable absorbent undergarments manufactured today resemble regular cloth underwear in that they have a waist opening and a pair of leg openings. Such pant-like disposable absorbent undergarments can be pulled up around the torso of a wearer in a similar fashion as regular cloth underwear. Still other pant-like disposable absorbent undergarments have an open or flat configuration and are designed to be placed adjacent to a wearer's torso and then rely upon one or more attachment tabs or fasteners to secure the undergarment around the wearer's torso. This design is beneficial for bed bound users who may be immobile or for babies or children who need assistance in securing the undergarment in place. Still other adjustable, pant-like absorbent undergarments contain attachment means for opening and closing the waist opening after the undergarment has been positioned around the wearer's torso. This type of adjustable undergarment has an advantage in that the wearer does not have to remove outer clothing in order to check the status of the undergarment or to remove the undergarment from their body.

One example of such an adjustable, pant-like disposable absorbent undergarment includes a pair of lines of weakness that a user must break to enable adjusting the fit of the undergarment. The lines of weakness usually extend from the waist opening to one of the leg openings and are designed to be broken either prior to positioning the undergarment around the user's torso or while the undergarment is already positioned around the wearer's torso. A pair of fastener assemblies or attachment tabs is then utilized to refasten the undergarment so that it is snug about the wearer's torso.

It has been found that a major portion of each of the lines of weakness is visually hidden and some users cannot see them and thereby do not know that they are present. In addition, each line of weakness may be ergonomically hard to tear open by older adults, some of who may be suffering from arthritis or other physical ailments. In addition, options that require tearing often indicate to users that the product is damaged or of poor quality when torn.

An adjustable, pant-like disposable absorbent undergarment that includes a fully severed front panel and more readily apparent and more easily accessible fastener mechanisms is needed. Typically, however, the types of consumer goods mentioned above are manufactured on a continuous basis on large scale manufacturing lines. Usually, various raw products or components are formed on, or integrated into, a continuous stream of material, which often includes a web of material that moves in a machine direction through and along the line. As such, it is important to maintain the integrity of the stream of material or web during the process so as to avoid costly downtime. In general, the web is pushed or pulled along the line, so as to put the web in tension. Accordingly, the formation of a fully severed panel, especially along a cross-direction, can increase the risk of breakage. Therefore, it is desirable to maintain the tensile strength of the stream of materials or web as it passes through the process.

Therefore, there is need to provide a method and apparatus for manufacturing pant-like disposable absorbent undergarment that includes a fully severed front panel without causing a problem with the integrity of the stream of material or web during the process.

### SUMMARY

Generally, a method and apparatus for producing a pre-fastened adjustable pant-like disposable absorbent undergarment including a fully severed front body panel is disclosed. The method includes the steps of providing a moving web and first creating a line of weakness on the moving web at a weakening station to define a leading portion of the moving web and a trailing portion of the moving web connected at the line of weakness. The moving web then passes through an attachment assembly station to bridge the connected leading portion and trailing portion together with a fastener assembly that extends over the line of weakness. Finally, the moving web is then passed through a web breaking station to completely separate the leading portion and trailing portion of the moving web at the line of weakness, such that the leading portion and trailing portion remains bridged together by the fastener assembly.

In exemplary embodiments, the web breaking station comprises providing an insert member, and pushing the insert member into at least a portion of the moving web near the line of weakness with the insert member into the recess causing the leading portion and trailing portion to separate at the line of weakness.

In one exemplary embodiment, the web breaking station comprises first and second moveable members, wherein the first moveable member comprises at least one insert member and wherein the second moveable member comprises at least one recess shaped to receive the insert member, and further comprising pushing into at least a portion of the moving web near the line of weakness with the insert member into the recess causing the leading portion and trailing portion to separate at the line of weakness. In this embodiment, it is desirable for the insert member to extend at least 75% of a length of the line of weakness.

In another exemplary embodiment, the web breaking station comprises a first contact device adapted to receive the leading portion of the web and a second contact device adapted to receive the trailing portion of the web. In this embodiment, the first contact device and the second contact device move in relation to each other causing the leading portion and trailing portion to separate at the line of weakness. The first contact device and the second contact device may move in relation to each other in variety of different ways. For example the first contact device may rotate from the second contact device, the first contact device may pivot from the second contact device, and the first contact device may separate from the second contact device.

In some embodiments, to enable the web breaking station to break the web, the fastener assembly is attached to the web in a flexible position. For example, the fastener assembly may be stretchable and placed on the moving web in a relaxed condition. Alternatively, the fastener assembly may be applied in a folded configuration or as a loop of a material.

### BRIEF DESCRIPTION

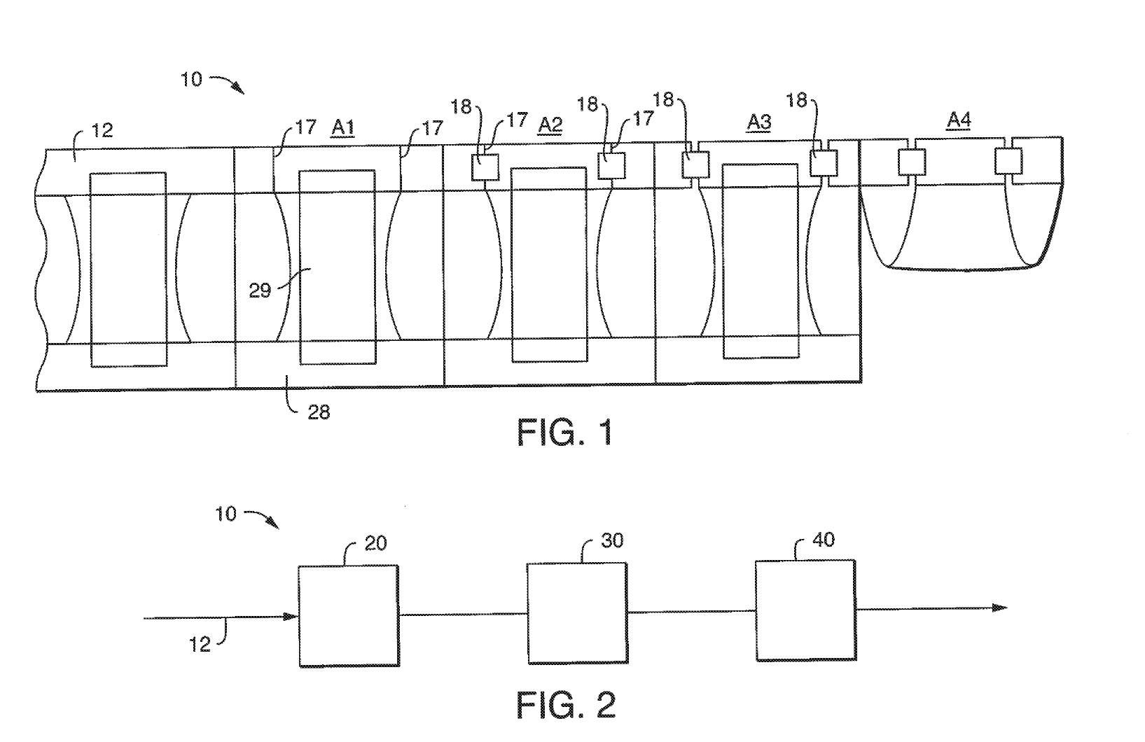

FIG. 1 depicts a method of producing a pre-fastened adjustable pant-like disposable absorbent undergarment including a fully severed front body panel.

FIG. 2 depicts an apparatus for producing a pre-fastened adjustable pant-like disposable absorbent undergarment including a fully severed front body panel.

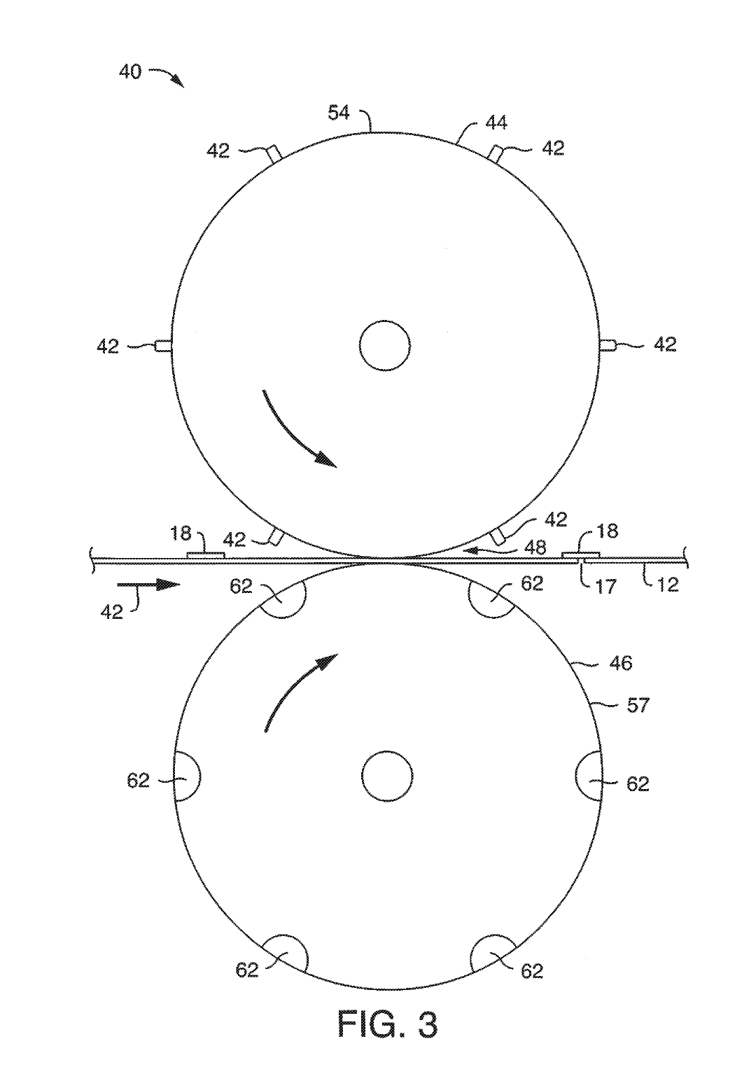

FIG. 3 depicts an exemplary embodiment of a web-breaking station for use of the method in FIGS. 1 and 2.

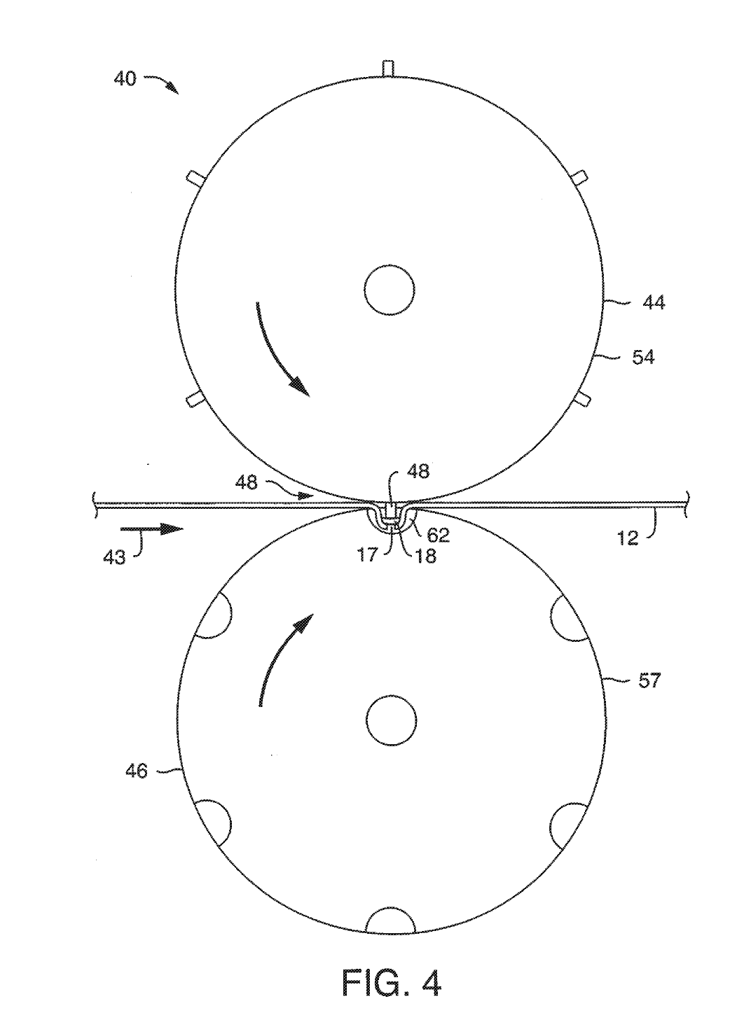

FIG. 4 depicts a close-up view of the exemplary embodiment of a web-breaking station in FIG. 3.

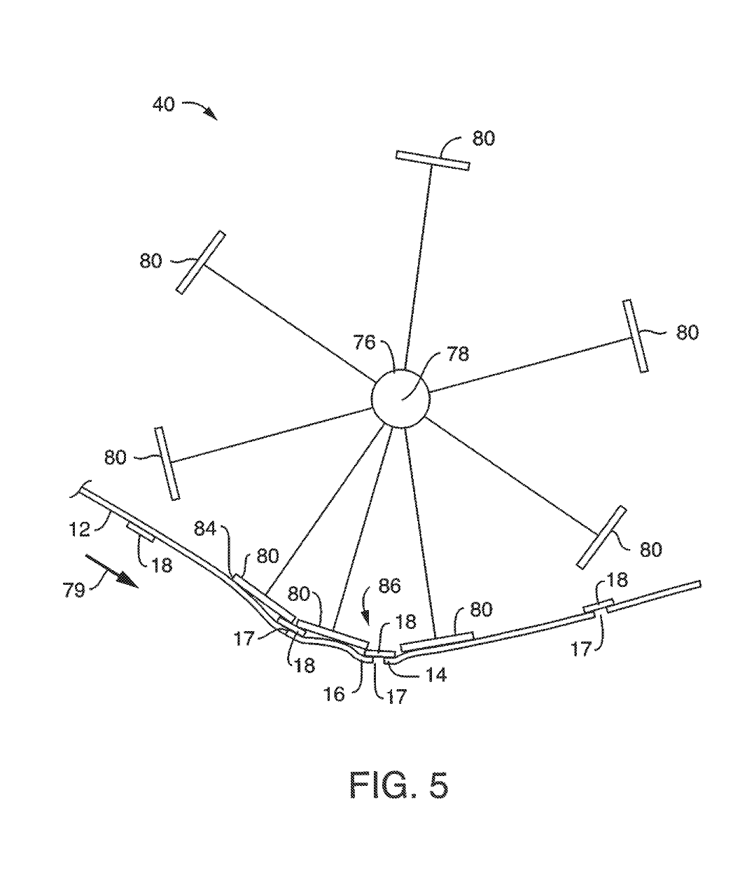

FIG. 5 depicts another exemplary embodiment of a web-breaking station for use of the method in FIGS. 1 and 2.

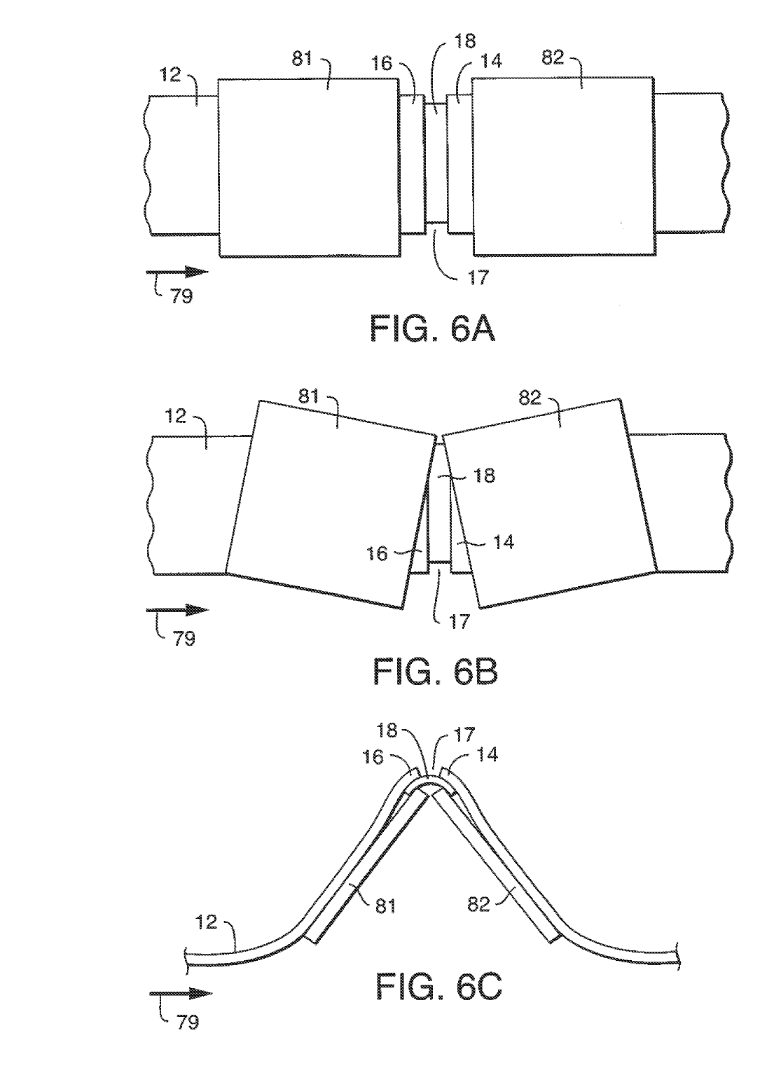

FIG. 6A-6C depict exemplary embodiments of the contact devices for use at the web breaking station in FIG. 5.

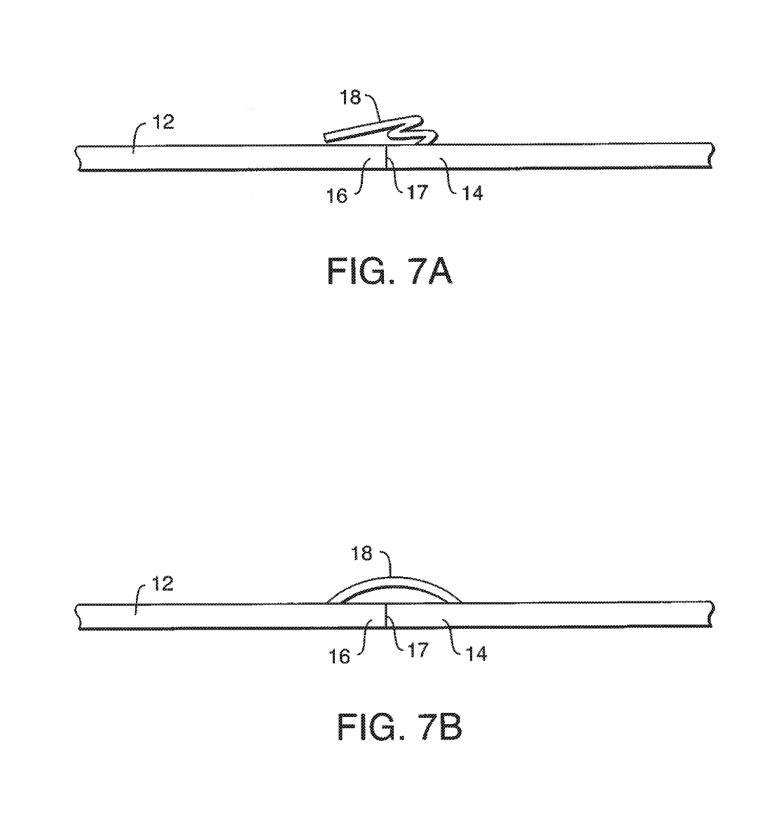

FIGS. 7A-7B depict exemplary embodiments of placement of the fastener for use of the method and apparatus disclosed herein.

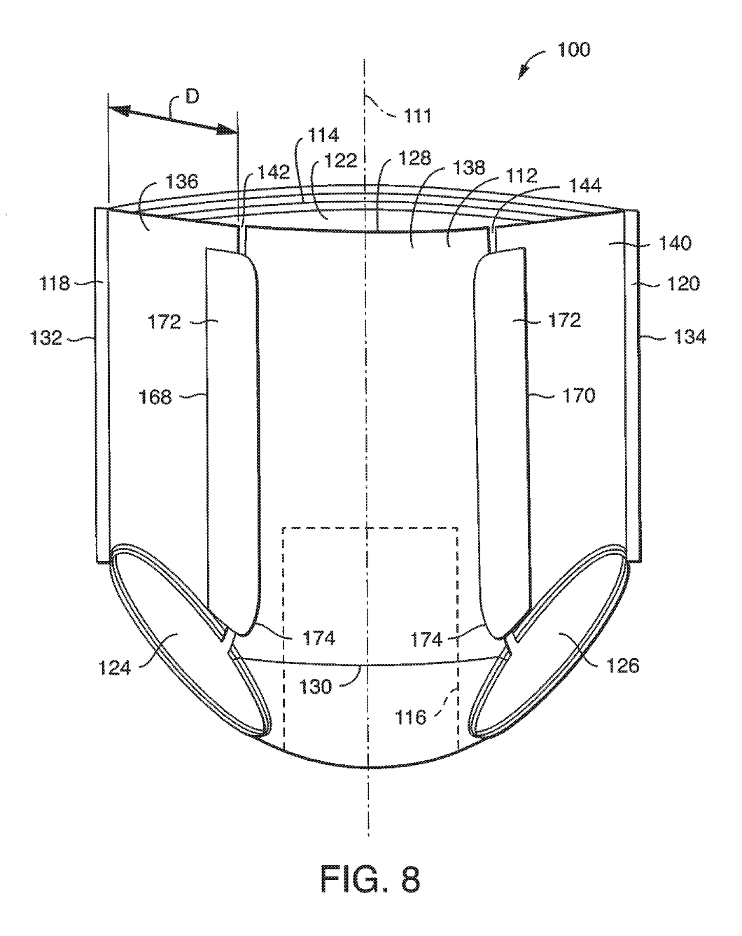

FIG. 8 depicts a front perspective view of one example of a garment, the garment shown in a pre-fastened, pant-like configuration.

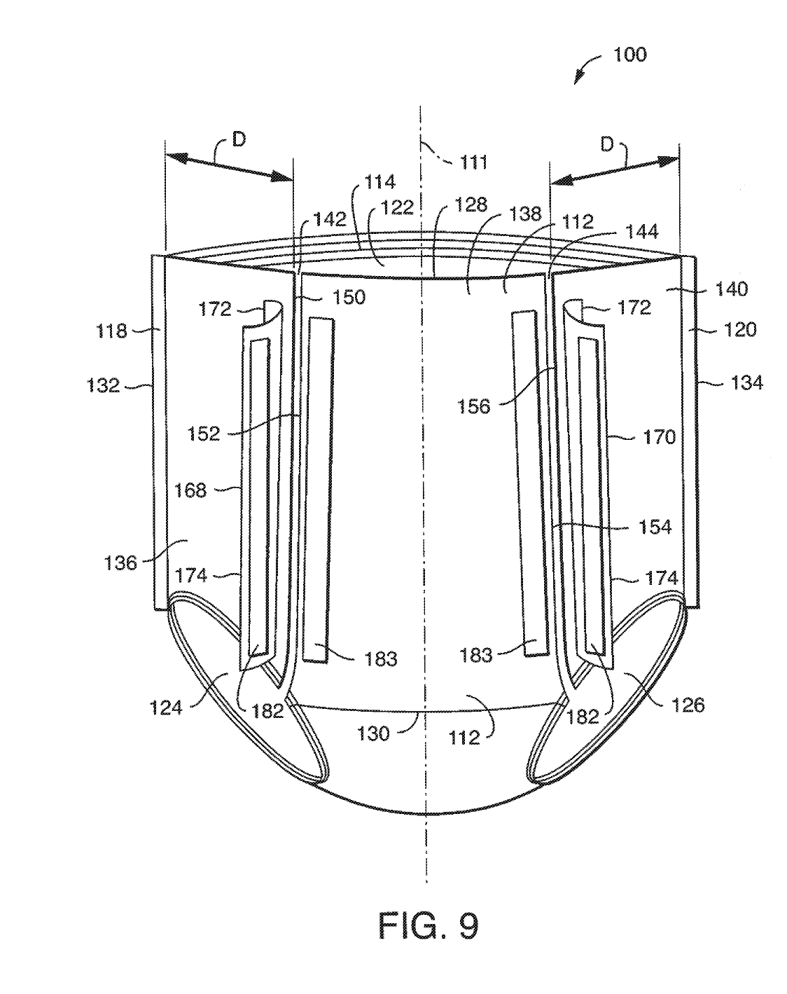

FIG. 9 depicts a front perspective view of one example of a garment, the garment shown in an unfastened, pant-like configuration.

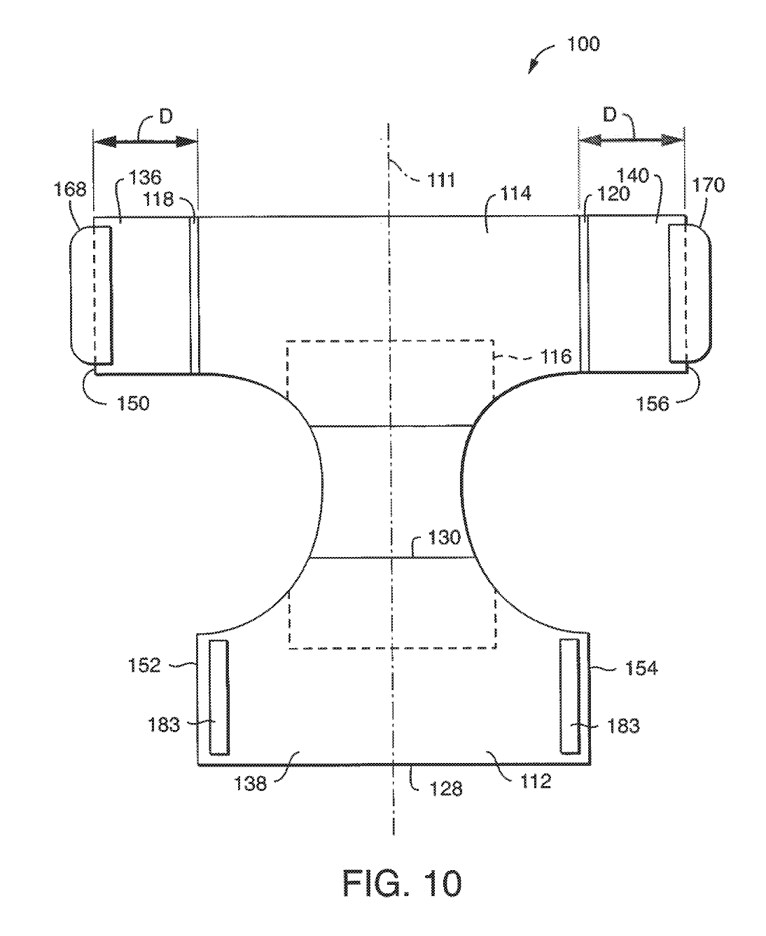

FIG. 10 depicts a plan view of the garment of FIG. 1, the garment shown in an unfastened, laid-open, relaxed configuration.

### DETAILED DESCRIPTION

Reference to the Figures shall be made in describing various embodiments. It should be noted that the embodiments depicted in the Figures and described herein are merely representative examples. The various embodiments are suitable for use in conjunction with disposable absorbent undergarments such as refastenable adult incontinence underwear, pre-fastened disposable diapers, refastenable disposable training pants or swim pants, refastenable disposable enuresis garments, and the like. For illustration purposes, various embodiments shall be described in conjunction with refastenable incontinence or enuresis underwear.

Within the context of this specification, each term or phrase below will include the following meaning or meanings.

The term “body side” should not be interpreted to mean in contact with the body of the user, but rather simply means the side that would face toward the body of the user when the garment is applied to the user, regardless of whether the absorbent garment is actually being worn by the user and regardless of whether there are or may be intervening layers between the component and the body of the user. Likewise, the term “garment side” should not be interpreted to mean in contact with the garments of the user, but rather simply means the side that faces away from the body of the user when the garment is applied to the user, and therefore toward any outer garments that may be worn by the user, regardless of whether the absorbent garment is actually being worn by a user, regardless of whether any such outer garments are actually worn and regardless of whether there may be intervening layers between the component and any outer garment.

The term “machine direction” means the direction of flow as the various members and webs progress along the fabrication line and process. It should be understood that various separate members or webs can each be traveling in a machine direction, but with the various machine directions not necessarily being parallel or oriented in the same direction. For example, one web may be traveling along a first machine direction, which is substantially perpendicular to the travel of another web in a second machine direction.

The term “cross direction” means the direction substantially perpendicular to the machine direction.

The term “downstream” means that one item is positioned more closely to the output or finished product end of the machine and/or process relative to another item. Conversely, the term “upstream” means that an item is positioned more closely to the input end of the machine or process relative to another item. For example, the output end is downstream of the input end, and vice versa, the input end is upstream of the output end.

The term “disposable absorbent undergarment” as used herein is an article that is intended to be worn by persons, including infants, toddlers or adults, which is designed for a single or temporary use and is meant to be disposed of after being used once instead of being laundered or dry cleaned for re-use.

The term “attached” refers to the joining, adhering, bonding, connecting, or the like, of two elements. Two elements will be considered to be attached together when they are attached directly to one another or indirectly to one another, such as when each is directly attached to intermediate elements.

The term “disposable” refers to articles which are designed to be discarded after a limited use rather than being laundered or otherwise restored for re-use.

The term “elastomeric” refers to a material or composite which can be elongated by at least 50% of its relaxed length and which will recover, upon release of the applied force, at least 20% of its elongation. It is generally preferred that the elastomeric material or composite be capable of being elongated by at least 100%, more preferably by at least 200%, of its relaxed length and recover, upon release of an applied force, at least 50% of its elongation.

The term “stretchable” refers to a material or composite which can be elongated by at least 5% of its relaxed length without breaking with or without recovery. It is generally preferred that the stretchable material or composite be capable of being stretchable by at least 10%, more preferably by at least 20% or more, of its relaxed length without breaking.

The term “weakening” means to cause to lose strength, such that the area that is weakened is not as strong as the adjacent areas. For example, and without limitation, an area that is weakened may have a lesser tear or tensile strength as compared with the adjacent areas of the web, such that the web is more likely to be torn or broken along the area of weakness rather than the adjacent areas. In this way, the manufacturer can control the area of the web that will be broken, whether such breakage is performed by the end user or at a later time during the manufacturing or fabrication process.

The term “line of weakness” refers to any region or area of weakened material, preferably having a length and which may or may not have a defined width, and can include linear and non-linear patterns, such as curvilinear patterns of weakness, or other shapes, such as a circles, rectangles, etc. The line of weakness can include a perforation or other series of cuts, a thinning, or breakage or separation of material, or a strip of a different kind of material bridging between adjacent portions of material, that is more easily torn or broken than the adjacent portions, and which allow the user or manufacturer to separate the adjacent portions along the line of weakness.

“Longitudinal” and “transverse” have their customary meaning, as indicated by the longitudinal and transverse axes depicted in the Figures. The longitudinal axis lies in the plane of the article and is generally parallel to a vertical plane that bisects a standing wearer into left and right body halves when the article is worn. The transverse axis lies in the plane of the article generally perpendicular to the longitudinal axis.

These terms may be defined with additional language in the remaining portions of the specification.

Generally, a method and apparatus for producing a pre-fastened adjustable pant-like disposable absorbent undergarment including a fully severed front body panel is disclosed. The method includes providing a moving web that is used to produce a front body panel. The method includes the steps of providing a moving web and first creating a line of weakness on the moving web at a weakening station to define a leading portion of the moving web and a trailing portion of the moving web connected at the line of weakness. The moving web then passes through an attachment assembly station to bridge the connected leading portion and trailing portion together with a fastener assembly that extends over the line of weakness. Finally, the moving web is then passed through a web breaking station to completely separate the leading portion and trailing portion of the moving web at the line of weakness, such that the leading portion and trailing portion remains bridged together by the fastener assembly.

Referring to FIGS. 1 and 2, a method and apparatus for producing a pre-fastened adjustable pant-like disposable absorbent undergarment is illustrated. A moving web 12 is shown as moving in a machine direction along a process line. The moving web 12 may be used as a front body panel, a back body panel, or both in the disposable absorbent undergarment.

The moving web 12 can be formed from a single piece of material or can be formed as a laminate consisting of two or more layers. The layers of the laminate can be of the same material or different material. In one embodiment, a laminate is formed from a first layer and a second layer. Sandwiched between the first and second layers are two or more elastic strands. Desirably, from two to about a hundred elastic strands can be utilized in the moving web 12 depending upon the overall size of each panel. The elastic strands can be formed from LYCRA, or a similar material. LYCRA is a trademark of INVISTA (Wichita, Kans.). The diameter and/or cross-sectional configuration of the elastic strands, the decitex (weight in grams per 10,000 meters) of the elastic strands, and the tension imparted into the elastic strands can all be varied to suit a particular product needs. The exact number of elastic strands that are utilized should be sufficient to ensure that the disposable absorbent undergarment 10 snuggly conforms to the wearer's torso.

The elastic strands can be coated with an adhesive. By adhesively coating each of the elastic strands, instead of slot coating a major portion of the inner surface of at least one of the first and second layers, softer moving webs 12 respectively, can be obtained. Wearers of disposable absorbent undergarments prefer a product that has a softer feel since it is more like underwear.

In other embodiments, the elastomeric nonwoven material comprises an elastomeric film sandwiched between two nonwoven facing layers. U.S. Pat. No. 7,803,244 to Siqueira et al., hereby incorporated by reference, discloses particular examples of elastomeric nonwoven composites suitable for use in the disposable adjustable undergarment described herein.

It should be noted that the moving web 12 can be formed from a breathable or a non-breathable material. Desirably, the moving web 12 is formed from a breathable material or a material that is treated or processed to be breathable. Spunbond and bonded carded webs are two breathable materials that work well to form a moving web 12 in disposable absorbent undergarments. Bonded carded webs are produced and commercially sold by a variety of vendors. Other materials that can be used to form the moving web 12 include woven and non-woven materials formed from natural or synthetic fibers; polyolefins, such as polypropylene or polyethylene; thermoplastic films; as well as other materials known to those skilled in the art. A metallocene polypropylene works very well since it has a soft feel and can be easily ultrasonically bonded to itself.

The moving web 12 is first passed through a weakening station 20 at a first position A1. The weakening station 20 forms a line of weakness 17 in the moving web 12 to define a leading portion 14 of the moving web 12 and a trailing portion 16 of the moving web 12 connected at the line of weakness 17, as shown for example in FIGS. 1 and 2. In a desirable embodiment, the weakening station 20 forms a cross-direction line of weakness 17, which is preferably linear, in the moving web 12. The line of weakness 17 can extend across the entire cross-direction width of the moving web 12, or along only a portion thereof. In some embodiments, the line of weakness 17 can be tapered relative to the longitudinal axis 111, if desired. In addition, the line of weakness 17 can also be curved.

In one desirable embodiment, the first weakening station 20 is configured as a perforator having a knife roll and an anvil roll. Alternatively, the weakening station 20 can be configured with a laser, water jet, or other types of cutters known to those skilled in the art. In other alternative embodiments, the weakening station 20 can comprise a device for applying heat, thermal energy or ultrasonic energy to the web so as to weaken it at specific locations, or lines of weakness. In other preferred embodiments, the weakening station 20 can include a chemical applicator that applies various chemicals, including for example water, to the web to weaken it at specific locations. In yet another alternative embodiment, the apparatus applies a speed differential to the web so as to weaken the moving web 12. Of course, it should be understood that the weakening station 20 can also be configured from combinations of one or more of the above-referenced devices.

Downstream from the weakening station 20, the moving web 12 is passed through a fastener assembly attachment station 30 at a second position A2. The fastener assembly attachment station 30 attaches a fastener assembly 18 that bridges the connected leading portion 14 of the moving web 12 and trailing portion 16 of the moving web 12 together with a fastener assembly 18 that extends over the line of weakness 17. The fastener assembly 18, which may be applied soon after the lines of weakness 17 are formed, maintains the integrity of the moving web 12 as it continues through the process. The fastener can be securely attached using an adhesive, heat, pressure, a combination of heat and pressure, an ultrasonic bond, a chemical bond or by other means known to those skilled in the art. The fastener may also be a refastenable mechanical fastener.

Further downstream from the fastener assembly attachment station 30, the moving web 12 is passed through a web breaking station 40 at a third position A3. The web breaking station 40 completely separates the leading portion 14 of the moving web 12 and trailing portion 16 of the moving web 12 at the line of weakness 17, such that the leading portion 14 and trailing portion 16 remains bridged together by the fastener assembly 18.

In one desirable embodiment, the web breaking station 40 includes an insert member 42 that protrudes from a surface and pushes into the moving web 12, the web moving in the direction of arrow 43, adjacent or near the line of weakness 17 so as to completely separate leading 14 and trailing portions 16 of the moving web 12 along the line of weakness 17. For examples, intermeshed gears could be used wherein a gear would push into the web and cause the web to break at the line of weakness 17.

In one exemplary embodiment as illustrated in FIGS. 3 and 4, such a web breaking station 40 includes first and second moveable members or rolls 44, 46 forming a nip 48 through which the web passes. The first roll 44 is preferably configured as a knife roll, with the second roll 46 preferably configured as an anvil roll. The first and second rolls 44, 46 rotate in opposite directions about first and second longitudinal axes respectively. The first roll 44 has an outer surface 54 and a plurality of insert members 42 extending outwardly from the outer surface 54, and preferably extending radially outward from the outer surface. Preferably, a plurality, meaning two or more, insert members 42 are spaced around the periphery of the roll 44. Preferably, the insert members 42 are positioned so as to be registered with the lines of weakness 17 formed in the web 12 by the weakening station 20 as the lines of weakness 17 are passing through the nip 48. Preferably, the insert members 42 have a length extending along a cross direction parallel to the longitudinal axis of the first roll 44.

The insert member 42 may be configured as an elongated bar. Preferably, the insert member 42 has the length that is at least 75% of the length of the line of weakness 17 and therefore functions to break the entire line of weakness 17. It should be understood that in an alternative embodiment, the insert member 42 can be configured simply as a flat bar having an edge that extends beyond the outer surface 54 of the first roll 44.

Referring to FIGS. 3 and 4, the second roll 46 has an outer surface 54 and a plurality of recesses 62 formed and extending inwardly from the outer surface 54. Preferably, the recesses 62 extend radially inward from the outer surface 54 and are circumferentially spaced so as to mate with and receive the insert member 42 of the first roll 44 at the nip 48 formed between the two rolls 44, 46, as shown for example in FIG. 4. In addition, the recesses 62 are preferably formed along the cross-direction in the longitudinal direction and have a length dimensioned to receive the insert members 42. While illustrated as the first roll 44 having the insert members 42 and the second roll 46 having recesses 62, it is also possible wherein the first roll 44 includes the recesses 62 and the second roll 46 includes the insert members 42.

In one preferred embodiment, the two rolls 44, 46 cooperate to break the line of weakness 17 as at least a portion of the web 12 is forced by the insert members 42 into the recesses 62 formed in the second roll 46 so as to completely separate leading and trailing portions 14, 16 of the web 12 along the line of weakness 17.

In another embodiment, the web breaking station 40 includes a first contact device 80 adapted to receive the leading portion 14 of the moving web 12 and a second contact device 80 adapted to receive the trailing portion 16 of the moving web 12. Once the moving web 12 is in contact with the web breaking station 40, the first contact device 80 and the second contact device 80 move in relation to each other causing the leading portion 14 and trailing portion 16 to completely separate at the line of weakness 17.

In one suitable embodiment illustrated in FIG. 5, the web breaking station 40 includes a drive assembly 76 configured to rotate about a drive axis 78, and a plurality of contact devices 80 coupled to and extend outward from the drive assembly. The drive assembly 76 includes one or more suitable drive sources including, for example, servo motors, and/or camboxes, operatively connected to each of the contact devices 80. More specifically, the drive source(s) of the drive assembly 76 is configured to rotate each contact device 80 about the drive axis 78 between a pick-up location, indicated generally at 84, and dropoff location, indicated generally at 86. In the illustrated embodiment, the drive assembly 76 is configured to rotate each of the contact devices 80 in a counter clockwise direction.

As illustrated in FIG. 5, each of the contact devices 80 is configured to receive the web 12 moving in the direction of arrow 79, at the pick-up location 84, and convey the moving web 12 to the web-breaking location 40. The first contact device 80 engages with the leading portion 14 of the web 12 and the second contact device 80 engages with the trailing portion 16 of the moving web 12. As illustrated in FIG. 5, two of the contact devices 80 are disposed in close proximity to each other as the moving web 12 is initially received by the web breaking station 40.

During each full rotation of the web breaking station 40, each of the contact devices 80 rotates along a first variable speed profile. In other words, the contact devices 80 of the web breaking device 40 rotate at variable speeds during each revolution. For example, each of the contact devices 80 rotates at a first speed (as measured at the outer surface of the contact device 80). Preferably, the first speed of the contact devices 80 approximately matches the speed at which the web 12 is being fed to the web breaking station 40.

After the leading portion 14 of the web 12 is received by the first contact device and the trailing portion 16 of the moving web 12 is received by a second contact device, the drive assembly 76 accelerates the first contact device to a second, faster speed. As a result, the accelerated first contact device separates from the adjacent, trailing second contact device. Thus, the two contact devices that were adjacent at the pick-up location 84 are separated which causes the moving web 12 to break about the preformed line of weakness 17 and thereby separate the leading portion 14 of the web 12 from the trailing portion 16 of the moving web 12.

As illustrated in FIG. 5, each contact device includes a leading edge 14, a trailing edge 16, and a platform that extends between the leading edge 14 and the trailing edge 14. The platform of each of the contact devices 80 is sized and shaped to receive and hold the moving web 12. The moving web 12 can be held to the contact devices 80 by vacuum, surface roughness and/or needle/clamp engagement or other suitable means known to those skilled in the art.

As illustrated in FIG. 6A-C, the first contact device 81 and the second contact device 82 may move in relation to each other in a variety of different ways causing the leading portion 14 and trailing portion 16 to completely separate at the line of weakness 17. For example, as illustrated in FIG. 6A, the first contact device 81 from the second contact device 82 may separate away from each other to cause the line of weakness 17 to break. Alternatively, as illustrated in FIG. 6B, the first contact device 81 from the second contact device 82 may pivot away from each other causing the line of weakness 17 to break. In still another embodiment as illustrated in FIG. 6C, the first contact device 81 may rotate from the second contact device 82, causing the line of weakness 17 to break. Additionally, a combination of any of the above techniques to move the contact devices 81, 82 may be used to cause the moving web 12 to break.

In some embodiments, to enable the web breaking station 40 to break the moving web 12, the fastener assembly 18 is flexible or extensible. For example, the fastener assembly 18 may be stretchable. The fastening assembly 18 is placed on the moving web 12 in a relaxed condition. The moving web 12 is in a stretched out condition at this point in the process. When the seam on the moving web 12 is stressed the stretchable fastener assembly 18 gives without breaking. Alternatively, a fastener assembly 18 with or without stretch could be attached to the moving web 12 in a flexible position. The fastener assembly 18 may be applied in a folded configuration as illustrated in FIG. 7A or in a loop of a material configuration as illustrated in FIG. 7B while the moving web 12 is in its about stretched out condition. When the moving web 12 is stressed to break the weaken area, the flexible condition will provide give in the fastener assembly 18 without breaking.

Attaching the fastener assembly 18 in a flexible position while the moving web 12 is in a stretched out position provides a disposable undergarment 10 having a body panel wherein the leading portion 14 of the moving web 12 becomes a first section of the body panel and the trailing portion 16 of the moving web 12 becomes a second section of the body panel. When the moving web 12 is broken between leading and trailing portions 14, 16, the body panel is completely severed between the first section and the second section. In this embodiment, the fastener assembly 18 is attached to the first section of the body panel and attached to the second section of the body panel. As the process is finished, the moving web 12 is relaxed from the stretched out condition and therefore contracts. Accordingly, the length of material of the fastener assembly 18 between where the fastener assembly 18 is attached to the first section of the body panel and the second section of the body panel has a first distance. Since the moving web 12, and thus body panel, has contracted, the length of the material of the body panel between where the fastener assembly 18 is attached to the first section of the body panel and the second section of the body panel has a second distance. Desirably, the second distance is less than the first distance.

Various methods and apparatus for manufacturing absorbent garments and for applying fastener members thereto are disclosed in U.S. Pat. No. 6,730,188 entitled “Method and Apparatus For Assembling Refastenable Absorbent Garments,” U.S. Pat. No. 6,743,321 entitled “Method and Apparatus For Assembling Refastenable Absorbent Garments,” U.S. Pat. No. 6,686,626 entitled “Method and Apparatus For Assembling Refastenable Absorbent Garments,” U.S. Pat. No. 6,682,626 entitled “Method and Apparatus For Assembling Refastenable Absorbent Garments,” U.S. Pat. No. 6,712,922 entitled “Multiple Component Web,” U.S. Pat. No. 6,730,188 entitled “Method of Assembling Personal Care Absorbent Article,” U.S. Pat. No. 6,783,487, entitled “Pant-Type Personal Care Articles, and Methods of Making and Using Such Personal Care Articles,” U.S. Pat. No. 6,454,888 entitled “Methods of Changing Size of Pant-Type Personal Care Articles Outputted from a Manufacturing Process,” the entire disclosures of which are hereby incorporated by reference.

In one embodiment, illustrated in FIG. 1, at a fourth position A4, the moving web 12 is used as a front body panel web and bonded to a rear body panel web 28 at side seams, wherein the rear body panel web 28 is positioned over the moving web 12 by folding a crotch portion 29, joining or bridging between the moving web 12 and the back body panel web 28. This may be done prior to or after the moving web 12 is introduced to the web breaking station 40.

Referring to FIGS. 8-10, an adjustable pant-like disposable absorbent undergarment 100 having a longitudinal axis 111 made using the method described herein is shown. The adjustable pant-like disposable absorbent undergarment 100 is designed to absorb liquid, semi-solid and/or solid waste discharged from a human being. The adjustable pant-like disposable absorbent undergarment 100 is designed to absorb and/or retain one or more bodily discharges of waste material such as urine, perspiration, excrement, feces, menses, menstrual fluid, as well as other liquid and/or solid waste.

The adjustable pant-like disposable absorbent undergarment 100 includes a front body panel 112, a back body panel 114 and an absorbent assembly 116 secured to the front and back body panels, 112 and 114 respectively. The front and back body panels, 112 and 114 respectively, are joined together by a pair of seams 118 and 120 to form a waist opening 122 and a pair of leg openings 124 and 126.

The front body panel 112 includes a waist edge 128, a crotch edge 130 and a pair of side edges 132 and 134. In a three piece construction wherein an absorbent assembly 116 is secured between the front body panel 112 and the back body panel 114, the crotch edge 130 is well defined. In absorbent undergarments of a different construction, the crotch edge 130 can be an imaginary line transversely drawn between the pair of leg openings 124 and 126 at a location where one considers the front body panel 112 to end. The exact size and configuration of the front body panel 112 can vary to suit a wearer's particular needs. The front body panel 112 has a first side section 136, a middle region 138 and a second side section 140. The front body panel 112 is completely severed between both the first side section 136 and the middle section 138 and the second side section 140 and the middle section 138 creating a first fully severed region, or gap, 142 and a second fully severed region 144. The first side section 136 is aligned adjacent to the side edge 132 at its leading edge forming the seam 120 and the second side section 140 is aligned adjacent to the side edge 134 at its leading edge forming the seam 118. The middle section 138 has a first edge 132 and a second edge 134 and is located between the first and second sections, 136 and 140 respectively. The terminal edge 150 of the first side section 136 is aligned adjacent to the first edge 152 of the middle section 138 and the terminal edge 150 of the second side section 140 is aligned adjacent to the second edge 154 of the middle section 138. As depicted, the middle section 138 is centrally located and is bifurcated by the longitudinal axis 111 at a midpoint of the front body panel 112.

The first and second fully severed regions, 142 and 144, can be linear or non-linear in configuration. In FIGS. 8-10, the first and second fully severed region, 142 and 144, are shown having a linear or straight configuration. The first and second fully severed regions, 142 and 144, extend longitudinally from approximately the waist edge 128 down to approximately one of the leg openings 124 or 126. The first and second fully severed regions, 142 and 144, can be aligned parallel to the longitudinal axis 111 or be angled thereto. For example, the first and second fully severed regions, 142 and 144, can be tapered relative to the longitudinal axis 111, if desired. In addition, the fully severed regions, 142 and 144, can also be curved.

Still referring to FIGS. 8-10, each of the first and second fully severed regions, 142 and 144, extend from approximately the waist edge 128 of the front body panel 112 to one of the pair of leg openings 124 and 126. Another way of describing this is to say that the first and second fully severed regions, 142 and 144, extend from approximately the waist edge 128 of the front body panel 112 to approximately the crotch edge 130 of the front body panel 112.

Many adjustable, pant-like disposable absorbent undergarments include a pair of lines of weakness to break to allow for the individual to adjust the size of the pants. This type of adjustable disposable undergarment including the lines of weakness requires an amount of force needed to break the lines of weakness. The pre-fastened adjustable, pant-like disposable absorbent undergarments 100 described herein have the first and second fully severed regions, 142 and 144, that require no force to break allowing for easier use by an elderly individual, caretaker or parent caring for a child.

Still referring to FIGS. 8-10, each of the first and second fully severed regions, 142 and 144, is shown being aligned parallel to one of the pair of seams 118 and 120. Such an arrangement provides for an aesthetically pleasing appearance to the front body panel 112.

Desirably, in the pre-fastened condition, the first side sections 136 and the middle section 138 of front body panel 112 do not overlap, and the second side section 140 and the middle section 138 of the front body panel 112 do not overlap in the severed regions 142, 144. In one exemplary embodiment, a first gap may be formed in the first severed region 142 between the first side section 136 and the middle section 138 of the front body panel 112 in the pre-fastened condition. In this embodiment, a second gap may be formed in the first severed region 142 between the second side section 140 and the middle section 138 of the front body panel 112 in the pre-fastened condition. In another exemplary embodiment, a terminal or outer edge 150 of the first side section 136 abuts against the first edge 152 of the middle section 138 of front body panel 112 in the pre-fastened condition. In this embodiment, a terminal or outer edge 150 of the second side section 140 abuts against the second edge 154 of the middle section 138 of the front body panel 112 in the pre-fastened condition. In another embodiment, a gap may be formed between one side section and the middle section 138 and the other side section and middle section 138 abut against each other. If a gap is formed between the side sections 136 and 140, and the middle section 138, the gap will desirably be less than 10 mm.

Referring again to FIGS. 8-10, the front body panel 112 further includes a pair of fastener assemblies 168 and 170. Each fastener assembly 168 and 170 includes a first portion 172 and a second portion 174. The first portion 172 of the fastener assembly 168 can be permanently secured to the first side panel 136 of the front body panel 112 and the first portion 172 of the other fastener assembly 170 can be permanently secured to the second side panel 140 of the front body panel 112.

In the embodiment shown in FIGS. 8-10, the first portions 172 of the fastener assemblies 168 and 170 can be securely attached using an adhesive, heat, pressure, a combination of heat and pressure, an ultrasonic bond, a chemical bond or by other means known to those skilled in the art.

Each of the second portions 174 of the fastener assemblies 168 and 170 are depicted as extending almost the entire length of the fully severed portions 142 and 144. This means at least 50% of the distance between the waist edge 128 and the leg opening 124, 126 are covered by the fastener assembly 18. Desirably, the first fastener assembly 168 and second fastener assembly 170 extend at least 65% of the distance between the waist edge 128 and the leg opening 124, 126. More desirably, the first fastener assembly 168 and second fastener assembly 170 extend at least 75% of the distance between the waist edge 128 and the leg opening 124, 126. Even more desirably, the first fastener assembly 168 and second fastener assembly 170 extend at least 85% of the distance between the waist end and the leg opening. Even more desirably, the first fastener assembly and second fastener assembly completely extend at least 95% of the distance between the waist edge 128 and the leg opening 124, 126. The second portions 174 of the fastener assembly 168 can bridge across the first fully severed region 142 and the second portion 174 of the other fastener assembly 170 can bridge across the second fully severed region 144. By extending substantially the entire length of and bridging the fully severed portions, 142 and 144, the fasteners 168, 170 cover the fully severed portions 142, 144 and provide a more underwear like appearance. The second portions 174 of the fastener assemblies 168 and 170 can be releasably attached to the middle section 138 of the front body panel 112.

Alternatively, it should be evident to those skilled in the art that the first portion 172 of each of the fastener assemblies 168 and 170 could be permanently attached to the middle section 138. In this embodiment, the second portions 174 of the fastener assemblies 168 and 170 can be releasably attached to the side sections of the front body panel 112. In an alternative embodiment, both the first portion 172 and the second portion 174 may be releasably attached. The fastener 116 may be constructed of a non-extensible, stretchable, or elastomeric material.

Referring now to FIGS. 9 and 10, each of the second portions 174 of the fastener assemblies 168 and 170 has an inner surface 180 that contains a fastener 182. The fastener 182 can be a mechanical fastener 182. In FIGS. 9 and 10, the mechanical fastener 182 is shown as a plurality of fine hooks, such as VELCRO hooks. VELCRO is a trademark of Velcro USA, Inc. (Manchester, N.H.). The hooks are designed to easily engage and be removed from a material wherein the material has a loose weave pattern or the fibers forming the material will allow the hooks to be attached to them. The mating material is commonly referred to as the loop member of a hook and loop fastener. The middle section 138 of the front body panel 112 may be formed of such a material. When the hooks engage into the middle section 138, a secure but releasable fastener is formed. The hooks can be easily removed from the loop material by pulling the edge of the fastener outward away from the middle section 138. Alternatively, as illustrated in FIGS. 9 and 10, a fastening component 183 such as a loop material or loose fibers that may be placed anywhere onto the middle section 138 to facilitate the fastening component. Therefore, hook and loop fasteners are referred to as being releasable and can be fastened and released several times. In alternative embodiments, the mechanical fastener 182 constructed of hooks may be placed on to the middle section 138 of the front body panel 112. In this embodiment, the fastening component 183 constructed of a loop material or loose fibers would be placed on the fastener assemblies 168, 170. Both the mechanical fastener 182 and the fastening component 183 may be integral or separately attached.

Referring again to FIGS. 8-10, one can see that FIG. 8 depicts the pair of fastener assemblies 168 and 170 being securely fastened to the middle section 138 of the front body panel 112. If the wearer of the disposable absorbent undergarment 100 or a caregiver wishes to inspect the undergarment 100, he or she would open the pair of fastener assemblies 168 and 170 to the position shown in FIG. 2. Since the first and fully severed portions, 142 and 144 are already broken, the middle section 138 of the disposable absorbent undergarment 100 can be easily moved outward away from the wearer's torso. The wearer can then inspect the absorbent assembly 116 to see if it needs to be changed. If so, the disposable absorbent undergarment 100 can be removed from about the wearer's torso and be replaced by another undergarment. If the absorbent assembly 116 is still capable of accepting additional body fluid, the middle section 138 is moved back against the wearer's torso and the pair of fastener assemblies 168 and 170 is refastened to the middle section 138.

The horizontal distance to each of the first and second fully severed regions, 142 and 144, and corresponding first and second fastener assemblies, 168 and 170, as located relative to the pair of side seams 118 and 120 can also vary. A distance “D” is depicted in FIGS. 8-10 which represents the distance between each of the pair of seams 118 and 120 and the corresponding first and second fully severed regions, 142 and 144 respectively. One can increase the distance “D” by moving the first and second fully severed regions, 142 and 144 respectively, away from the pair of seams 118 and 120. When one increases the distance “D”, one may find that it is easier for the wearer of the disposable absorbent undergarment 100 to visually see and identify the first and second fully severed regions, 142 and 144, when user looks down at the front body panel 112. In some embodiments, the first fastener assembly 168 and the second fastener assembly 170 are located near the midpoint between the side seams, 118 and 120, and the cross-sectional midpoint 111 of the front body panel 112. Desirably, the first fastener assembly 68 and second fastener assembly 70 are located between 30% and 70% of the distance between the side seam, 118 and 120, and the cross-sectional midpoint 111 of the front panel 112. More desirably, the first fastener assembly 168 and second fastener assembly 170 are located between 35% and 45% of the distance between the side seams, 118 and 120, and the cross-sectional midpoint 111 of the front panel 112. A manufacturer is free to vary the distance “D” to best suit the size and shape of a particular disposable absorbent undergarment 100 to make sure that the fastener is on the front of the product rather than the side of the product. If the fully severed regions are not parallel to the side seam, the midpoint of the fully severed regions in the longitudinal direction may be used to measure “D”.

Placement near the midpoint between the side seam 118 and the cross-sectional midpoint 111 of the front body panel 112 facilitates enhanced donning and removal when experiencing a reduced range of motion as well as locating the fastener in a location which is easier for the consumer to see.

The placement of the fastener assemblies 168 and 170 in the front of the product optimizes the ability of the user to fasten the product, while minimizing potential pop-opens during use due to movement of the legs of the user. The placement of the fasteners can be measured in both an unstretched (out of the bag) state or stretched (as in use).

It should be understood that various other embodiments, modifications, and equivalents to the embodiments of the absorbent article described herein which, after reading the description herein, may suggest themselves to those skilled in the art without departing from the scope and spirit of the present claims.

What is claimed is:

1. A method of producing pre-fastened disposable garments, comprising:
a. providing a moving web for use as a body panel;
b. forming a line of weakness on the moving web to define a leading portion of the moving web and a trailing portion of the moving web connected at the line of weakness;
c. bridging the connected leading portion of the moving web and trailing portion of the moving web together with a fastener assembly that extends over the line of weakness;
d. completely separating the leading portion of the moving web and trailing portion of the moving web at the line of weakness, such that the leading portion and trailing portion remains bridged together by the fastener assembly.

2. The method of claim 1 forming the line of weakness at the first location comprises perforating the moving web.

3. The method of claim 1 wherein completely separating the leading portion and trailing portion at the line of weakness comprises providing an insert member, and pushing the insert member into at least a portion of the moving web near the line of weakness with the insert member into a recess shaped to receive the insert member causing the leading portion and trailing portion to separate at the line of weakness.

4. The method of claim 3 wherein completely separating the leading portion and trailing portion at the line of weakness comprises moving the web between a first moveable member and a second moveable member, wherein the first moveable member comprises at least one insert member extending from the first moveable member, and wherein the second moveable member comprises at least one recess shaped to receive the insert member, and further comprising pushing into at least a portion of the moving web near the line of weakness with the insert member into the recess causing the leading portion and trailing portion to separate at the line of weakness.

5. The method of claim 4 wherein said insert member extends at least 75% of a length of the line of weakness.

6. The method of claim 1 wherein completely separating the leading portion and trailing portion at the line of weakness comprises providing a first contact device adapted to receive the leading portion of the web and a second contact device adapted to receive the trailing portion of the web; and
moving the first contact device and the second contact device in relation to each other causing the leading portion and trailing portion to separate at the line of weakness.

7. The method of claim 6 wherein moving the first contact device and the second contact device in relation to each other is selected from rotating the first contact device from the second contact device, pivoting the first contact device from the second contact device, and separating the first contact device from the second contact device.

8. The method of claim 1 wherein the fastener assembly is attached to the web in a flexible position.

9. The method of claim 1 wherein the fastener assembly is stretchable and placed on the moving web in a relaxed condition.

10. The method of claim 1 wherein the fastener assembly is attached to the web in a folded configuration or a loop of a material.

11. An apparatus for producing a pre-fastened adjustable pant-like disposable absorbent undergarment comprising:
a. a weakening station for forming a line of weakness on the moving web for use as a body panel to define a leading portion of the moving web and a trailing portion of the moving web connected at the line of weakness;
b. a fastener assembly attachment station for bridging the connected leading portion of the moving web and trailing portion of the moving web together with a fastener assembly that extends over the line of weakness; and
c. a web breaking station for completely separating the leading portion of the moving web and trailing portion of the moving web at the line of weakness, such that the leading portion and trailing portion remains bridged together by the fastener assembly.

12. The apparatus of claim 11 wherein forming the line of weakness at the first location comprises perforating the moving web.

13. The apparatus of claim 11 wherein the web breaking station comprises providing an insert member, and pushing the insert member into at least a portion of the moving web near the line of weakness with the insert member into a recess shaped to receive the insert member causing the leading portion and trailing portion to separate at the line of weakness.

14. The apparatus of claim 11 wherein the web breaking station comprises a first moveable member and a second moveable member, wherein the first moveable member comprises at least one insert member extending from said first moveable member and wherein the second moveable member comprises at least one recess shaped to receive the insert member, and further comprising pushing into at least a portion of the moving web near the line of weakness with the insert member into the recess causing the leading portion and trailing portion to separate at the line of weakness.

15. The apparatus of claim 14 wherein said insert member extends at least 75% of a length of the line of weakness.

16. The apparatus of claim 11 wherein the web breaking station comprises a first contact device adapted to receive the leading portion of the web and a second contact device adapted to receive the trailing portion of the web; and
wherein the first contact device and the second contact device move in relation to each other causing the leading portion and trailing portion to separate at the line of weakness.

17. The apparatus of claim 16 wherein moving the first contact device and the second contact device in relation to each other is selected from rotating the first contact device from the second contact device, pivoting the first contact device from the second contact device, and separating the first contact device from the second contact device.

18. The apparatus of claim 11 wherein the fastener assembly is attached to the web in a flexible position.

19. The apparatus of claim 11 wherein the fastener assembly is stretchable and placed on the moving web in a relaxed condition.

20. The apparatus of claim 11 wherein the fastener assembly is attached to the web in a folded configuration or a loop of a material.

21. A pre-fastened adjustable pant-like disposable absorbent undergarment comprising:
a. a body panel having a first section and a second section, wherein the body panel is completely severed between the first section and the second section;
b. a fastener assembly being attached to the first section and attached to the second section, the first fastener assembly being releasably attached to either said first section or second section, and wherein the length of material of the fastener assembly between where the fastener assembly is attached to the first side section and the second section has a first distance and the length of the material of the body panel between where the fastener assembly is attached to the first section and the second section has a second distance, wherein the second distance is less than the first distance.
